(19) United States
(12) Patent Application Publication
(10) Pub. No.: US 2010/0274003 A1
(43) Pub. Date: Oct. 28, 2010

(54) ACID ADDITION SALTS OF SYNTHETIC INTERMEDIATES FOR CARBAPENEM ANTIBIOTICS AND PROCESSES FOR PREPARING THE SAME

(75) Inventors: Chang-Seob Kim, Gyeonggi-do (KR); Dong-Woo Lee, Seoul (KR); Jae-Woo Lee, Seoul (KR); Mi-Ran Jung, Gyeonggi-do (KR); Young-Cheol Jeong, Gyeonggi-do (KR); Si-Min Kim, Gyeonggi-do (KR); Young-Ro Choi, Seoul (KR); Dong-Geun Seo, Incheon (KR); Ju-Hee Lee, Gyeonggi-do (KR); Phil-Sang Ahn, Incheon (KR)

Correspondence Address: THE NATH LAW GROUP, 112 South West Street, Alexandria, VA, 22314, US

(73) Assignee: Kukje Pharm Ind co. Ltd, Seongnam-si Gyeonnggi-do (KR)

(21) Appl. No.: 12/733,674

(22) PCT Filed: Sep. 17, 2008
(86) PCT No.: PCT/KR2008/005469

§ 371 Date: Mar. 15, 2010

(30) Foreign Application Priority Data
Sep. 20, 2007 (KR) 10-2007- 0095647
Sep. 20, 2007 (KR) 10-2007- 0095649

(51) Int. Cl. C07D 487/04

(52) U.S. Cl. 540/302

(57) ABSTRACT

The present invention provides a process for preparing an acid addition salt of a synthetic intermediate for carbapenem antibiotics and a novel acid addition salt of a synthetic intermediate for carbapenem antibiotics obtained from the process. The present invention also provides a process for preparing a carbapenem antibiotic using the acid addition salt. According to the process of the present invention, an acid addition salt of a synthetic intermediate for carbapenem antibiotics can be prepared in a high yield and high purity, without conducting column chromatography. Thus, the process of the present invention can be applied to mass production with an industrial scale. Furthermore, since the acid addition salts have solid forms, they are easy to handle and keep in a manufacturing site.

### TECHNICAL FIELD

The present invention relates to acid addition salts of synthetic intermediates for carbapenem antibiotics and processes for preparing the same. The present invention also relates to processes for preparing carbapenem antibiotics using the acid addition salts.

### BACKGROUND ART

With increase of infectious diseases around the world, use of antibiotics for the is treatment of the infectious diseases is being significantly increased. As a variety of antibiotics have been clinically used since 1980s, penicillin-resistant Streptococcus pneumoniae (PRSP), methicillin-resistant Staphylococcus aureus (MRSA), and other antibiotic-resistant bacteria cast problems over the world. Since vancomycin-resistant Enterococci (VRE) appeared in 1990s, the antibiotic-resistant bacteria have threatened human beings' health. Thus, there is a need to develop novel antibiotics capable of efficiently inhibiting antibiotic-resistant bacteria caused by misuse and overuse of antibiotics.

Thienamycin isolated from Streptomyces Cattleya by Merck Co., U.S.A. in 1976 is the first carbapenem antibiotic which is classified as a 4^{th} generation antibiotic. The carbapenem antibiotic has activity against β-lactamase-bearing strains due to its excellent antibiotic activity and broad antibiotic spectrum. Research on the synthesis of carbapenem antibiotics, as derivatives of thienamycin, has been conducted since the discovery of thienamycin. As a result, a variety of derivatives such as imipenem and meropenem have been developed and commercially available.

1-Beta-methylcarbapenem antibiotics are known as antibiotics having a broad spectrum of antibacterial activities against Gram-negative and Gram-positive bacteria (U.S. Pat. Nos. 4,962,103, 4,933,333, 4,943,569, and 5,122,604). Recently, it was reported that 2-arylcarbapenem compounds (L-695256 and L-742728) as carbapenem antibiotics showed good activity against MRSA and vancomycin-resistant Staphylococcus aureus (VRSA) (Hugh rosen et al., Sciences, 703 (1999)). WO 99/62906 discloses that 2-benzothiazolethenyl carbapenem has good activity against MRSA.

Carbapenem antibiotics have been clinically administered by injection due to low intestinal absorption and low storage stability thereof. L-084 (Meiji Corporation, Japan) has been developed as an oral carbapenem antibiotic having high intestinal absorption, and clinical tests thereof are being conducted (U.S. Pat. No. 5,534,510).

Meanwhile, the present inventors have developed a carbapenem antibiotic having excellent activity against MRSA and quinolone-resistant Staphylococcus aureus (QRSA) (Korean Patent No. 10-0599876). The carbapenem antibiotics developed by the present inventors are prepared according to Reaction Scheme 1 below:

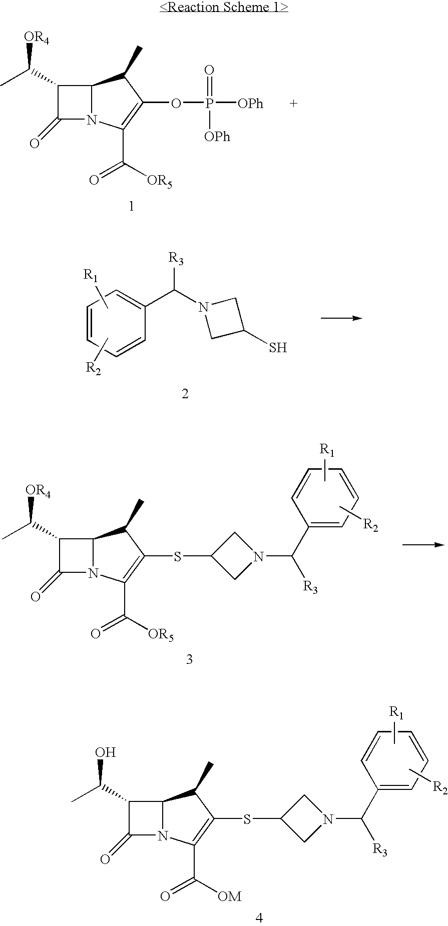

In Reaction Scheme 1, R₁ and R₂ are each independently hydrogen, C₁-C₃ alkyl, C₁-C₃ alkoxy, hydroxyl, amino, trifluoromethyl, or halogen; R₃ is hydrogen or C₁-C₃ alkyl; R₄ is hydrogen or a hydroxyl-protecting group; R₅ is carboxy-protecting group; and M is hydrogen or a counter ion forming a pharmaceutically acceptable salt.

As shown in Reaction Scheme 1, the intermediate, i.e., compound of Formula 3, is prepared by the reaction between compounds of Formulae 1 and 2. However, since column chromatography should be necessarily conducted in the final stage in order to purely obtain the compound of Formula 3, the process is not appropriate to mass production with an industrial scale. Furthermore, the yield of the product, i.e., the compound of Formula 3, obtained by the column chromatography is only about 40%. Furthermore, since the compound of Formula 3 is obtained in a liquid form, it is difficult to handle.

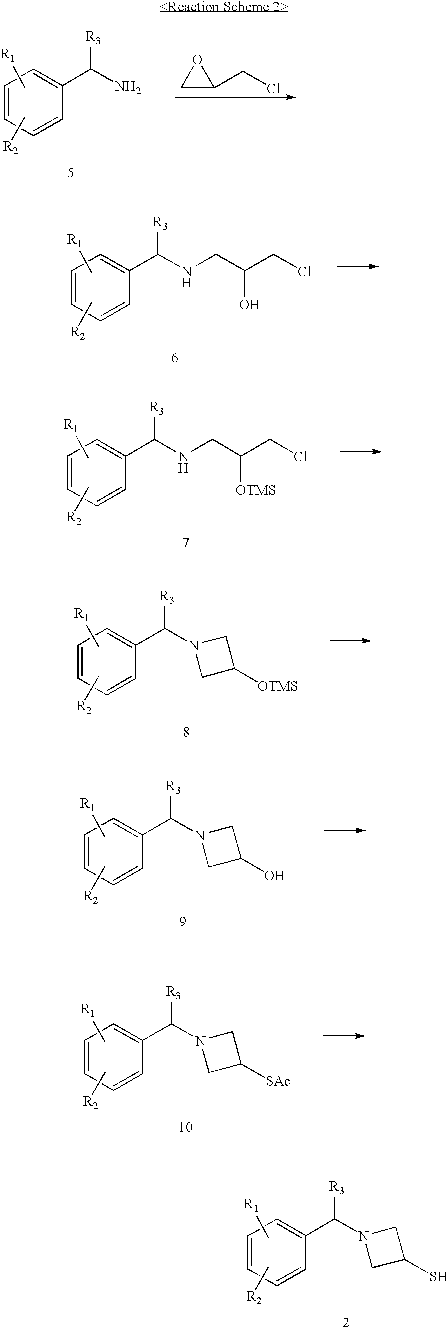

In Reaction Scheme 2, R₁ to R₃ are the same as defined in Reaction Scheme 1 above, TMS represents trimethylsilyl, and Ac represents acetyl.

As shown in Reaction Scheme 2 above, another intermediate, i.e., the compound of Formula 2, is prepared by conducting multi-step processes. That is, multi-step processes are necessary since the compound of Formula 9 is prepared via compounds of Formulae 7 and 8 obtained by silylation of the compound of Formula 6. In addition, since purification using column chromatography is necessary in the work-up stage in order to obtain the compound of Formula 10 from the compound of Formula 9, the yield of the product is low and the process is not appropriate to mass production with an industrial scale. Furthermore, since the compound of Formula 2 is obtained in a liquid form, it is also difficult to handle.

### DETAILED DESCRIPTION OF THE INVENTION

### Technical Problem

The present inventors conducted research on processes for preparing synthetic intermediates for carbapenem antibiotics in a high yield, without performing a process inappropriate for industrial application such as column chromatography.

As a result, the present inventors have found that an acid addition salt of a compound of Formula 3 can be prepared in a high yield and high purity, without performing column chromatography, by simply regulating the pH and crystallizing the compound of Formula 3 into an acid addition salt thereof in an organic solvent. Furthermore, since the acid addition salt of the compound of Formula 3 is obtained in a solid form, we found it easy to handle.

In addition, since an azetidine ring can be formed through the reaction between the compound of Formula 6 and a base without performing silylation, it is found that the process can be simplified. It was also found that the compound of Formula 10 can be simply prepared in a high yield and high purity, without performing column chromatography, by regulating pH to an acidic pH, neutralizing the resultant, and then crystallizing in an organic solvent, in the work-up stage of the compound of Formula 10. Furthermore, since the acid addition salt of the compound of Formula 2 is obtained in a solid form, we found it easy to handle.

Therefore, the present invention provides processes for preparing acid addition salts of synthetic intermediates for carbapenem antibiotics, in particular an acid addition salt of a compound of Formula 2 or a compound of Formula 3.

The present invention also provides an acid addition salt of the compound of Formula 2 or the compound of Formula 3.

The present invention also provides a process for preparing a compound of Formula 4 or a pharmaceutically acceptable salt thereof using the acid addition salt of the compound of Formula 3.

The present invention also provides a process for preparing a compound of Formula 9 from the compound of Formula 6 in a simple manner.

### Technical Solution

According to an aspect of the present invention, there is provided a process for preparing an acid addition salt of a compound of Formula 3, the process comprising: (a) reacting a compound of Formula 1 with a compound of Formula 2; (b) adding a mixed solvent of water and an organic solvent to the reaction mixture prepared in step (a), acidifying the resulting mixture to a pH ranging from 1 to 5, and then separating an organic layer; and (c) crystallizing by adding an organic solvent to the organic layer obtained in step (b) or its concentrate:

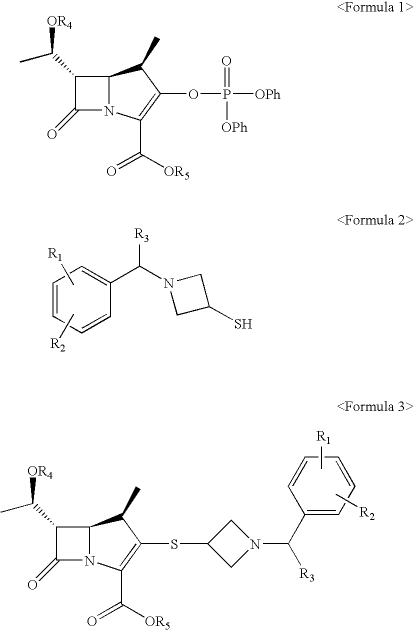

wherein, R₁ and R₂ are each independently hydrogen, C₁-C₃ alkyl, C₁-C₃ alkoxy, halogen, hydroxyl, amino, or trifluoromethyl; R₃ is hydrogen or C₁-C₃ alkyl; R₄ is hydrogen or a hydroxy-protecting group; and R₅ is a carboxy-protecting group.

According to another aspect of the present invention, there is provided an acid addition salt of the compound of Formula 3:

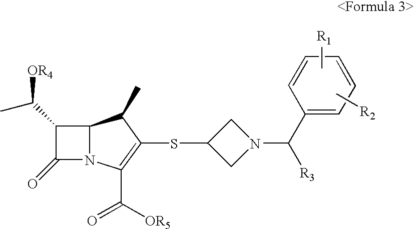

wherein, R₁, R₂, R₃, R₄, and R₅ are the same as defined in the above.

According to still another aspect of the present invention, there is provided a process for preparing a compound of Formula 4 or a pharmaceutically acceptable salt thereof, the process comprising deprotecting an acid addition salt of a compound of Formula 3:

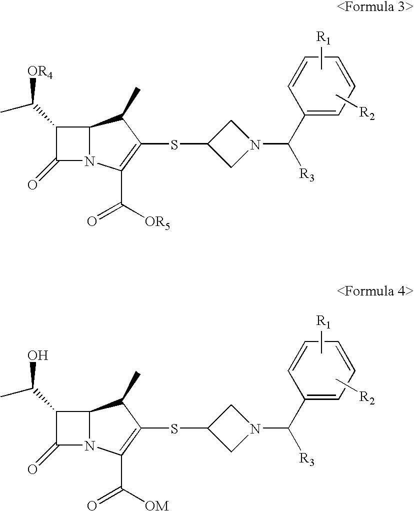

wherein, R₁, R₂, R₃, R₄, and R₅ are the same as defined in the above and M is hydrogen or a counter ion forming a pharmaceutically acceptable salt.

According to still another aspect of the present invention, there is provided a process for preparing an acid addition salt of a compound of Formula 2, the process comprising: (A) (i) reacting thioacetic acid with a compound of Formula 9, in the presence of triphenylphosphine and diisopropylazodicarboxylate or (ii) subjecting a compound of Formula 9, methanesulfonyl chloride, and alkali metal thioacetate to a reaction; (B) adding a mixed solvent of water and an organic solvent to the reaction to mixture prepared in step (A) or its concentrate, acidifying the resulting mixture to a pH ranging from 1 to 5, and then separating an aqueous layer; (C) neutralizing the aqueous layer obtained in step (B) and then extracting with an organic solvent to isolate a compound of Formula 10; (D) reacting the compound of Formula 10 obtained in step (C) with an inorganic base in C₁-C₄ alcohol to deacetylate the compound of Formula 10, and then forming an acid addition salt by adding a solution of an acid in C₁-C₄ alcohol; and (E) isolating the acid addition salt formed in step (D):

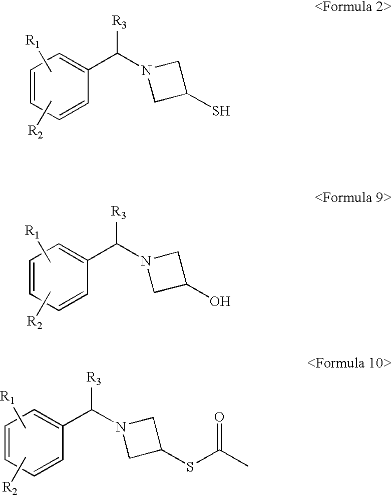

wherein, R₁ and R₂ are each independently hydrogen, C₁-C₃ alkyl, C₁-C₃ alkoxy, halogen, hydroxyl, amino, or trifluoromethyl; and R₃ is hydrogen or C₁-C₃ alkyl.

According to still another aspect of the present invention, there is provided a process for preparing a compound of Formula 6, the process comprising reacting a compound of Formula 5 with epichlorohydrin in water:

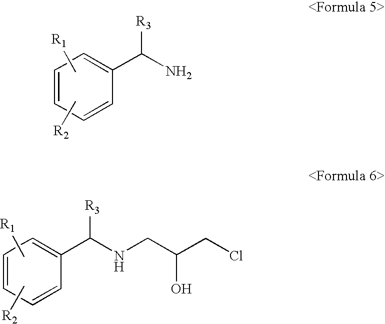

wherein, R₁, R₂ and R₃ are the same as defined in the above.

According to still another aspect of the present invention, there is provided a acid addition salt of the compound of Formula 2:

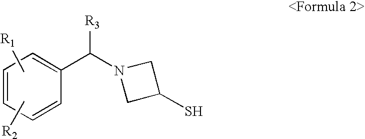

wherein, R₁, R₂, and R₃ are the same as defined in the above.

### ADVANTAGEOUS EFFECTS

According to the present invention, an acid addition salt of a compound of Formula 2 or a compound of Formula 3 can be respectively prepared in a high yield and high purity, without conducting column chromatography. Thus, the process of the present invention can be applied to mass production with an industrial scale. Furthermore, since the acid addition salts of the compounds of Formulae 2 and 3 have solid forms, they are easy to handle and keep in a manufacturing site.

A compound of Formula 4 or a pharmaceutically acceptable salt thereof can be prepared in a high yield and high purity by deprotection of an acid addition salt of the compound of Formula 3. In addition, the process can be simplified since an azetidine ring can be formed by the reaction between a compound of Formula 6 and a base, without conducting silylation.

### BEST MODE FOR CARRYING OUT THE INVENTION

The present invention includes a process for preparing an acid addition salt of a compound of Formula 3, the process comprising: (a) reacting a compound of Formula 1 with a compound of Formula 2; (b) adding a mixed solvent of water and an organic solvent to the reaction mixture prepared in step (a), acidifying the resulting mixture to a pH ranging from 1 to 5, and then separating an organic layer; and (c) crystallizing by adding an organic solvent to the organic layer obtained in step (b) or its concentrate:

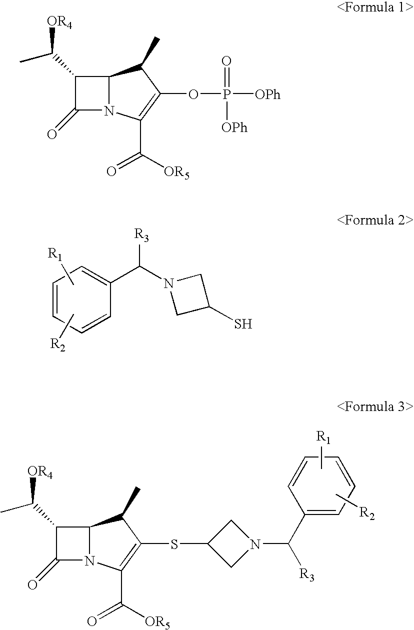

wherein, R₁ and R₂ are each independently hydrogen, C₁-C₃ alkyl, C₁-C₃ alkoxy, halogen, hydroxyl, amino, or trifluoromethyl; R₃ is hydrogen or C₁-C₃ alkyl; R₄ is hydrogen or a hydroxy-protecting group; and R₅ is a carboxy-protecting group.

In the process for preparing an acid addition salt of a compound of Formula 3 of the present invention, the hydroxy-protecting group may be conventional hydroxy-protecting groups such as tert-butyldimethylsilyl and triethylsilyl; and the carboxy-protecting group may be also conventional carboxy-protecting groups such as p-nitrobenzyl, allyl, and p-methoxybenzyl.

The step (a), i.e., the reacting a compound of Formula 1 with a compound of Formula 2, may be conducted according to the process disclosed in Korean Patent No. 10-0599876 developed by the present inventors. That is, the condensation between the compounds of Formulae 1 and 2 may be performed by dissolving the compound of Formula 2 in an anhydrous organic solvent, e.g., acetonitrile, methylene chloride, tetrahydrofuran, or acetone, preferably acetonitrile, cooling the solution to a temperature ranging from −20° C. to 0° C., slowly adding N,N-diisopropylethylamine or triethylamine to the resulting solution, adding the compound of Formula 1 thereto, and then stirring the resulting mixture at a temperature ranging from −20° C. to 0° C. for 2 to 4 hours.

The process of the present invention includes adding a mixed solvent of water and an organic solvent to the reaction mixture prepared in step (a), acidifying the resulting mixture to a pH ranging from 1 to 5, and then separating an organic layer [step (b)]. In the mixed solvent of water and the organic solvent, the ratio of water to the organic solvent may be 1:10 in equivalent ratio, preferably 1:1 in equivalent ratio, but is not limited thereto. The organic solvent may be ethyl acetate, tetrahydrofuran, methylene chloride, isopropyl ether, petroleum ether, or diethyl ether. The acidification to the pH ranging from 1 to 5, preferably to about pH 3, may be performed using conventional organic or inorganic acids, preferably using an inorganic acid such as hydrochloric acid, sulfuric acid, and phosphoric acid. The final form of the acid addition salt of the compound of Formula 3 is determined by the acid used for the acidification. That is, if hydrochloric acid is used for the acidification, a hydrochloride of the compound of Formula 3 is formed, and if sulfuric acid is used for the acidification, a sulfate of the compound of Formula 3 is formed.

The process of the present invention also includes crystallizing by adding an organic solvent to the organic layer obtained in step (b) or its concentrate [step (c)]. The organic layer obtained in step (b) may be directly used in the crystallization of step (c). If desired, the concentrate (i.e., residue) obtained by concentrating the organic layer using a conventional method, e.g., concentrating under a reduced pressure, may be used in the crystallization of step (c). The organic solvent used in the crystallization may be ethyl acetate, acetone, toluene, n-hexane, or isopropyl ether, preferably ethyl acetate or acetone. In the crystallization, the product, i.e. the acid addition salt of the compound of Formula 3, may be purely obtained by filtering, washing, and drying.

The acid addition salt of the compound of Formula 3 prepared according to the process of the present invention, as a novel substance, may be efficiently used in the synthesis of a carbapenem antibiotic of a compound of Formula 4 or a pharmaceutically acceptable salt thereof. Thus, the present invention provides the acid addition salt of the compound of Formula 3:

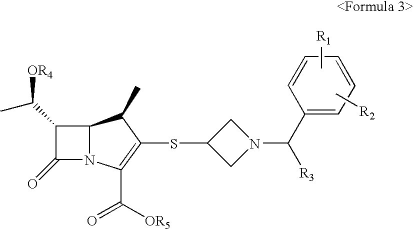

wherein, R₁, R₂, R₃, R₄, and R₅ are the same as defined in the above.

The acid addition salt may be an inorganic acid salt, preferably a hydrochloride, a sulfate, or a phosphate.

The present invention includes, within its scope, a process for preparing a carbapenem antibiotic of a compound of Formula 4 or a pharmaceutically acceptable salt thereof from the acid addition salt of the compound of Formula 3. According to Korean Patent No. 10-0599876, the compound of Formula 3 in the free base form is used as an intermediate for the preparation of the compound of Formula 4 or the pharmaceutically acceptable salt thereof. However, as described above, since the compound of Formula 3 in the free base form is in a liquid form, it is difficult to handle. Furthermore, it is surprisingly found that the compound of Formula 4 or the pharmaceutically acceptable salt thereof can be prepared with high purity of 98% or to greater by performing deprotection of the acid addition salt of the compound of Formula 3, without performing additional purification processes.

Thus, the present invention provides a process for preparing a compound of Formula 4 or a pharmaceutically acceptable salt thereof, the process comprising deprotecting an acid addition salt of a compound of Formula 3:

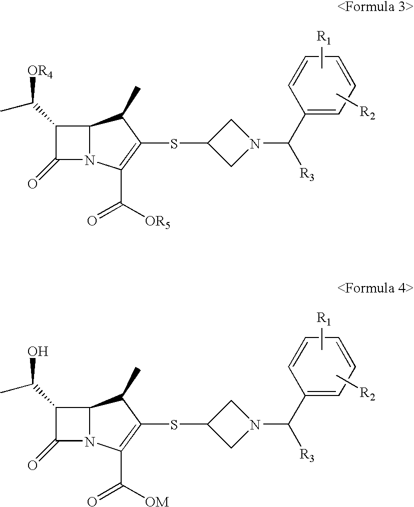

wherein, R₁, R₂, R₃, R₄, and R₅ are the same as defined in the above and M is hydrogen or a counter ion forming a pharmaceutically acceptable salt.

In the process for preparing the compound of Formula 4 or the pharmaceutically acceptable salt thereof, the acid addition salt of the compound of Formula 3 prepared as described above may be preferably used, and the acid addition salt of the compound of Formula 3 may be preferably a hydrochloride, a sulfate, or a phosphate. The deprotection may be conducted in the same manner as described in Korean Patent No. 10-0599876.

The present invention also includes a process for preparing an acid addition salt of a compound of Formula 2, the process comprising: (A) (i) reacting thioacetic acid with a compound of Formula 9, in the presence of triphenylphosphine and diisopropylazodicarboxylate or (ii) subjecting a compound of Formula 9, methanesulfonyl chloride, and alkali metal thioacetate to a reaction; (B) adding a mixed solvent of water and an organic solvent to the reaction mixture prepared in step (A) or its concentrate, acidifying the resulting mixture to a pH ranging from 1 to 5, and then separating an aqueous layer; (C) neutralizing the aqueous layer obtained in step (B) and then extracting with an organic solvent to isolate a compound of Formula 10; (D) reacting the compound of Formula 10 obtained in step (C) with an inorganic base in C₁-C₄ alcohol to deacetylate the compound of Formula 10, and then forming an acid addition salt by adding a solution of an acid in C₁-C₄ alcohol; and (E) isolating the acid addition salt formed in step (D):

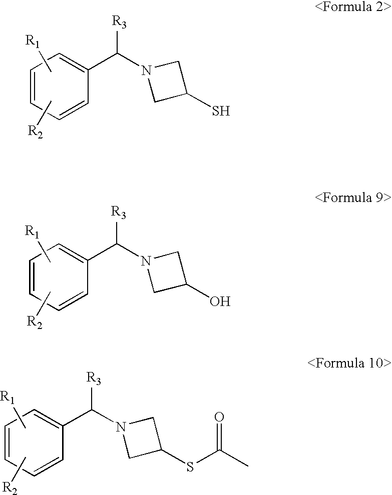

wherein, R₁, R₂ and R₃ are the same as defined in the above.

The step (A) may be performed by (i) reacting thioacetic acid with a compound of Formula 9, in the presence of triphenylphosphine and diisopropylazodicarboxylate or (ii) subjecting a compound of Formula 9, methanesulfonyl chloride, and alkali metal thioacetate to a reaction.

The reaction between thioacetic acid and the compound of Formula 9 in the presence of triphenylphosphine and diisopropylazodicarboxylate [i.e., step (i)] may be to performed in the same manner as described in Korean Patent No. 10-0599876, i.e. by applying the Mitsunobu reaction. For example, the reaction may be performed by adding diisopropylazodicarboxylate to a solution of triphenylphosphine in anhydrous tetrahydrofuran, reacting at 0° C. for about 1 hour, adding thioacetic acid and the compound of Formula 9 thereto, and then reacting at room temperature for 2 to 4 hours.

And also, the step (A) may be performed by subjecting a compound of Formula 9, methanesulfonyl chloride, and alkali metal thioacetate to a reaction [i.e., step (ii)]. It has been found, according to the present invention, that the reaction according to step (ii) may be performed at low cost using conventional manufacturing facilities, since the use of expensive diisopropylazodicarboxylate is not necessary and the anhydrous condition is not necessary.

The reaction of the compound of Formula 9, methanesulfonyl chloride, and alkali metal thioacetate [i.e., step (ii)] may be conducted by (p) reacting the compound of Formula 9 with methanesulfonyl chloride in the presence of a base; (q) adding water to the reaction mixture obtained in step (p), acidifying the resultant, separating an aqueous layer, adding an organic solvent to the aqueous layer, neutralizing the resultant, and then separating an organic layer; and (r) adding an organic solvent to the organic layer obtained in step (q) or its concentrate, and then reacting with alkali metal thioacetate

The base in step (p) may be triethylamine, trimethylamine, diisopropylethylamine, pyridine, 4-dimethylaminopyridine, guanidine, or the like. The amount of the base may be in the range of 1 to 15 equivalents, preferably 1 to 3 equivalents, based on 1 equivalent of the compound of Formula 9, but is not limited thereto. In addition, the reaction of step (p) may be performed in the presence of an organic solvent selected from the group consisting of methylene chloride, chloroform, ethyl acetate, tetrahydrofuran, acetone, acetonitrile, hexane, and toluene, preferably methylene chloride. In the reaction between the compound of Formula 9 and methanesulfonyl chloride of step (p), the amount of methanesulfonyl chloride may be in the range of 1 to 15 equivalents, preferably 1 to 3 equivalents, based on 1 equivalent of the compound of Formula 9.

In step (q), the acidification may be performed by regulating the pH to 1 to 5, preferably 1 to 3 using a hydrochloric acid solution, or the like. The organic solvent may be ethyl acetate, methylene chloride, chloroform, ethyl ether, petroleum ether, is toluene, hexane, or the like, preferably ethyl acetate.

In step (r), the organic solvent may be acetonitrile, chloroform, ethyl acetate, tetrahydrofuran, acetone, hexane, toluene, dimethylformamide, dimethylacetamide, or the like, preferably acetonitrile. The alkali metal thioacetate may be potassium thioacetate, sodium thioacetate, or the like, and the amount thereof may be in the range of 1 to 10 equivalents, preferably 1 to 2 equivalents, based on 1 equivalent of the compound of Formula 9.

The process of the present invention also includes adding a mixed solvent of water and an organic solvent to the reaction mixture prepared in step (A) or its concentrate, acidifying the resulting mixture to a pH ranging from 1 to 5, and then separating an aqueous layer [step (B)]. In the mixed solvent of water and the organic solvent, the ratio of water to the organic solvent may be 1:10 in equivalent ratio, preferably 1:1 in equivalent ratio, but is not limited thereto. The organic solvent may be ethyl acetate, methylene chloride, isopropyl ether, ethyl ether, petroleum ether, toluene, or n-hexane, preferably ethyl acetate (i.e., a mixed solvent of water and ethyl acetate). The acidification to the pH ranging from 1 to 5, preferably to the pH ranging from 3 to 4 may be performed using conventional organic or inorganic acids, preferably using an inorganic acid such as hydrochloric acid, sulfuric acid, and phosphoric acid.

The process of the present invention also includes neutralizing the aqueous layer obtained in step (B) and then extracting with an organic solvent to isolate a compound of Formula 10 [step (C)]. The neutralization may be performed by regulating the pH to about 7 to 8. The solvent used for the extraction may be ethyl acetate, methylene chloride, isopropyl ether, ethyl ether, petroleum ether, toluene, or n-hexane.

The process of the present invention also includes reacting the compound of Formula 10 obtained in step (C) with an inorganic base in C₁-C₄ alcohol to deacetylate the compound of Formula 10, and then forming an acid addition salt by adding a solution of an acid in C₁-C₄ alcohol [step (D)]. The inorganic base may be a conventional inorganic base such as sodium hydroxide (NaOH), potassium hydroxide (KOH), lithium hydroxide (LiOH), and calcium hydroxide (Ca(OH)₂), preferably NaOH or KOH. The amount of the inorganic base may be in the range of 1 to 3 equivalents, preferably 1.1 equivalent, based on 1 equivalent of the compound of Formula 10. The C₁-C₄ alcohol may be methanol, ethanol, isopropanol, or the like. The acid may be: an aliphatic acid such as acetic acid, propionic acid, butyric acid, trifluoroacetic acid, and trichloroacetic acid; a substituted or unsubstituted benzoic acid such as benzoic acid and nitrobenzoic acid; a low alkyl sulfonic acid such as methanesulfonic acid; an organic acid such as an organic phosphate such as diphenyl phosphate; or an inorganic acid such as hydrochloric acid, sulfuric acid, phosphoric acid, hydrobromic acid, hydroiodic acid, fluoroboric acid, perchloric acid, and nitrous acid, preferably hydrochloric acid, sulfuric acid or phosphoric acid. The acid may be used in the form of a C₁-C₄ alcoholic solution of the acid, for example, a solution of hydrochloric acid, sulfuric acid, or phosphoric acid in methanol.

The process of the present invention also includes isolating the acid addition salt formed in step (D) [step (E)]. The isolation of the acid addition salt may be performed by crystallizing the acid addition salt from isopropyl ether, ethyl acetate, or n-hexane.

As described above, the acid addition salt of the compound of Formula 2 prepared according to the present invention, as a novel substance, may be efficiently used as an intermediate for the synthesis of a carbapenem antibiotic of the compound of Formula 4 or the pharmaceutically acceptable salt thereof. Thus, the present invention provides the acid addition salt of the compound of Formula 2 below:

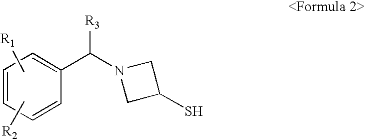

wherein, R₁, R₂, and R₃ are the same as defined in the above.

Preferably, the acid addition salt of the compound of Formula 2 may be a hydrochloride, a sulfate, or a phosphate.

In addition, the starting material (i.e., the compound of Formula 9) may be prepared by subjecting the compound of Formula 6 to silylation and desilylation as described in Korean Patent No. 10-0599876. According to the present invention, is however, it is newly found that the compound of Formula 9 may be prepared from the compound of Formula 6 using a single step without performing silylation. That is, the compound of Formula 9 may be prepared by reacting a compound of Formula 6 with at least one base selected from the group consisting of sodium bicarbonate (NaHCO₃), sodium carbonate (Na₂CO₃), potassium bicarbonate (KHCO₃), potassium carbonate (K₂CO₃), triethylamine (TEA), and diisopropylethylamine (DIPEA):

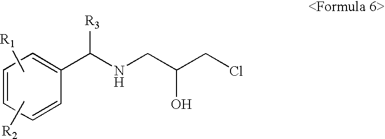

wherein, R₁, R₂ and R₃ are the same as defined in claim 1.

The amount of the base may be in the range of 1 to 5 equivalents, preferably 1.2 to 3 equivalents, based on 1 equivalent of the compound of Formula 6.

The reaction between the compound of Formula 6 and the base may be conducted by refluxing the reaction mixture in the presence of at least one solvent selected from the group consisting of acetonitrile, toluene, tetrahydrofuran, petroleum ether, and xylene at a refluxing temperature of the selected solvent. The reflux may be conducted for 8 to 24 hours, preferably 8 to 9 hours, and the reaction of azetidine cyclization shows purity of 98% or greater quantitatively.

The compound of Formula 6 may be prepared by reacting a compound of Formula 5 with epichlorohydrin in an organic solvent such as petroleum ether, as to described in Korean Patent No. 10-0599876. It has been found, according to the present invention, that problems caused by using the organic solvent such as petroleum ether, i.e., difficulty in the process management due to volatility of the organic solvent, toxicity of the organic solvent, environmental contamination, etc. may be solved by conducting the reaction of the compound of Formula 5 and epichlorohydrin in water. Therefore, the compound of Formula 6 may be preferably prepared by reacting a compound of Formula 5 with epichlorohydrin in water:

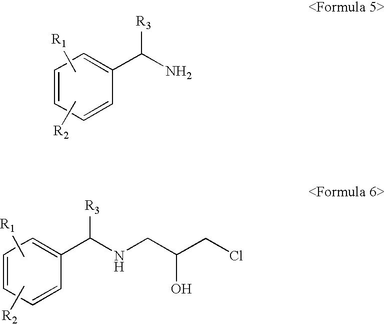

wherein, R₁, R₂ and R₃ are the same as defined in the above.

Hereinafter, the present invention will be described more specifically by the following working examples. However, the following working examples are provided only for illustrations and thus the present invention is not limited to or by them.

### Example 1

### 1-chloro-3-(4-fluorobenzylamino)propan-2-ol

35 Kg of 4-fluorobenzylamine was added to 500 Kg of purified water, and the reaction mixture was cooled to 5° C. or less. 38.8 Kg of epichlorohydrin was slowly added to the reaction mixture while the temperature of the reaction mixture is maintained at 5° C. or less. The reaction mixture was gradually heated to 20° C., stirred for 12 hours, and then filtered. Obtained solid was washed with hexane and dried under a reduced pressure to obtain 54.5 Kg of 1-chloro-3-(4-fluorobenzylamino)propan-2-ol (Yield: 89.5%).

¹H NMR (300 MHz, CDCl₃) δ 2.78 (m. 2H), 3.57 (d, J=5.3 Hz, 2H), 3.79 (s, 2H), 3.90 (m, 1H), 7.01 (m, 2H), 7.29 (m, 2H).

The compounds of Examples 2 to 7 were prepared in the same manner as in Example 1, except for using benzylamine, 4-methylbenzylamine, 4-methoxybenzylamine, 2-chlorobenzylamine, (1R)-1-phenylethylamine, or 3,4-dimethoxybenzylamine instead of 4-fluorobenzylamine.

### Example 2

### 1-chloro-3-benzylaminopropan-2-ol

Yield: 83%: ¹H NMR (300 MHz, CDCl₃) δ 2.72 (m, 2H), 3.56 (d, 2H), 3.80 (s, 2H), 3.86 (m, 1H), 7.32 (m, 5H).

### Example 3

### 1-chloro-3-(4-methylbenzylamino)propan-2-ol

Yield: 84%; ¹H NMR (300 MHz, CDCl₃) δ 2.23 (bs, 2H), 2.34 (s, 3H), 3.56 (d, 2H), 3.78 (d, 2H), 3.89 (m, 1H), 7.12 (d, 2H), 7.15 (d, 2H).

### Example 4

### 1-chloro-3-(4-methoxybenzylamino)propan-2-ol

Yield: 75%; ¹H NMR (300 MHz, CDCl₃) δ 2.79 (m, 2H), 3.55 (d, 2H), 3.76 (s, 2H), 3.80 (s, 3H), 3.91 (m, 1H), 6.86 (d, 2H), 7.24 (d, 2H).

### Example 5

### 1-chloro-3-(2-chlorobenzylamino)propan-2-ol

Yield: 78%; ¹H NMR (300 MHz, CDCl₃) δ 2.76 (m, 2H), 3.56 (m, 2H), 3.78 (s, 2H), 3.88 (m, 1H), 7.22 (m, 2H), 7.38 (m, 2H).

### Example 6

### 1-chloro-3-[(1R)-1-phenylethylamino]propan-2-ol

Yield: 80%; ¹H NMR (300 MHz, CDCl₃) δ 1.39 (d, 3H), 2.60 (m, 2H), 3.51 (m, 2H), 3.79 (m, 2H), 7.31 (m, 5H).

### Example 7

### 1-chloro-3-(3,4-dimethoxybenzylamino)propan-2-ol

Yield: 56%; ¹H NMR (300 MHz, CDCl₃) δ 2.72 (m, 2H), 3.50 (d, 2H), 3.91 (m, 9H), 6.80 (m, 3H).

### Example 8

### 1-(4-fluorobenzyl)azetidin-3-ol

1-Chloro-3-(4-fluorobenzylamino)propan-2-ol (500 g, 2.3 mol) prepared in Example 1 and sodium bicarbonate (482.4 g, 5.7 mol) were dissolved in acetonitrile (7 L). The reaction mixture was refluxed under stirring for 10 hours and cooled to room temperature. The reaction mixture was filtered to remove solid precipitates, and the filtrate was concentrated under a reduced pressure. The resulting residue was dissolved in ethyl acetate (1.5 L) and purified water (2.5 L) was added thereto. The pH of the resulting solution was adjusted to pH 2 using a diluted hydrochloric acid solution, and the resulting aqueous layer was separated. The separated aqueous layer was alkalized to pH 8 by adding 10% sodium hydroxide solution and extracted with ethyl acetate (2 L). Anhydrous magnesium sulfate was added to the extract, and the resulting solution was filtered to remove solid. The filtrate was concentrated under a reduced pressure to obtain 393 g of the pure title compound (Yield: 89.2%).

¹H NMR (300 MHz, CDCl₃) δ 2.91 (m, 2H), 3.57 (s, 2H), 3.58 (m, 2H), 4.43 (m, 1H), 7.03 (m, 2H), 7.23 (m, 2H).

The compounds of Examples 9 to 14 were prepared in the same manner as in Example 8, except for using the compounds prepared in Examples 2 to 7 instead of 1-chloro-3-(4-fluorobenzylamino)propan-2-ol.

### Example 9

### 1-benzylazetidin-3-ol

Yield: 88%: ¹H NMR (300 MHz, CDCl₃) δ 2.98 (m, 2H), 3.62 (m, 2H), 3.78 (m, 1H), 7.28 (m, 5H).

### Example 10

### 1-(4-methylbenzyl)azetidin-3-ol

Yield: 67%; ¹H NMR (300 MHz, CDCl₃) δ 2.32 (s, 3H), 2.92 (m, 2H), 3.56 (m, 4H), 4.41 (m, 1H), 7.09 (m, 4H).

### Example 11

### 1-(4-methoxybenzyl)azetidin-3-ol

Yield: 97%; ¹H NMR (300 MHz, CDCl₃) δ 2.91 (m, 2H), 3.53 (s, 2H), 3.57 (m, 2H), 3.78 (s, 3H), 4.41 (m, 1H), 6.83 (d, 2H), 7.15 (d, 2H).

### Example 12

### 1-(2-chlorobenzyl)azetidin-3-ol

Yield: 69%; ¹H NMR (300 MHz, CDCl₃) δ 2.92 (m, 2H), 3.56 (s, 2H), 3.60 (m, 2H), 4.41 (m, 1H), 7.22 (m, 2H), 7.36 (m, 2H).

### Example 13

### 1-[(1R)-1-phenylethyl]azetidin-3-ol

Yield: 67%; ¹H NMR (300 MHz, CDCl₃) δ 1.25 (d, 3H), 2.90 (m, 2H), 3.38 (m, 2H), 3.70 (m, 1H), 4.40 (m, 1H), 4.78 (bs, 1H), 7.27 (m, 5H).

### Example 14

### 1-(3,4-dimethoxybenzyl)azetidin-3-ol

Yield: 65%; ¹H NMR (300 MHz, CDCl₃) δ 2.88 (m, 2H), 3.55 (s, 2H), 3.58 (m, 2H), 3.85 (s, 3H), 3.86 (s, 3H), 4.39 (m, 1H), 6.78 (s, 2H), 6.81 (s, 1H).

### Example 15

### 1-(4-fluorobenzyl)-3-acetylthio-azetidine

Triphenylphosphine (188 g, 717 mmol) was dissolved in anhydrous tetrahydrofuran (2 L) under a nitrogen atmosphere, and the reaction mixture was cooled to 0° C. Diisopropylazodicarboxylate (141 ml, 717 mmol) was slowly added thereto, and the reaction mixture was stirred for 1 hour at the same temperature. Thioacetic acid (51 ml, 717 mmol) was slowly added to the reaction mixture under a nitrogen atmosphere, and the reaction mixture was stirred for 30 minutes. 1-(4-Fluorobenzyl)azetidin-3-ol (100 g, 552 mmol) prepared in Example 8 dissolved in tetrahydrofuran (275 ml) was slowly added to the reaction mixture. The reaction mixture was heated to room temperature, stirred for 2 hours, and then concentrated under a reduced pressure to remove the solvent completely. The resulting residue was dissolved in a mixed solution of ethyl acetate (1.5 L) and purified water (1 L). The resultant solution was acidified to pH 3.5 by adding a diluted hydrochloric acid solution, and then the aqueous layer was separated. An organic layer was extracted again with ethyl acetate (0.7 L) and then the aqueous layer was separated. The combined aqueous layer was neutralized to pH 7 with 10% sodium hydroxide solution, and then extracted with ethyl acetate (1 L×2). Anhydrous magnesium sulfate was added to the extract, which was then filtered. The filtrate was concentrated under a reduced pressure to obtain 109 g of the title compound (Yield: 82.5%).

¹H NMR (300 MHz, CDCl₃) δ 2.30 (s, 3H), 3.11 (m, 2H), 3.60 (s, 2H), 3.71 (m, 2H), 4.14 (m, 1H), 7.03 (m, 2H), 7.22 (m, 2H)

### Example 16

### 1-(4-fluorobenzyl)-3-acetylthio-azetidine

45.4 Kg of 1-(4-fluorobenzyl)azetidin-3-ol prepared according to Example 8 was dissolved in 550 Kg of methylene chloride. The reaction mixture was cooled to 5° C. or less, 38 Kg of triethylamine was added thereto, and then 34.3 Kg of methanesulfonyl chloride was slowly added thereto. The reaction mixture was stirred at a temperature ranging from 0 to 5° C. for 5 hours, and 385 Kg of purified water was added to the reaction mixture. The reaction mixture was acidified to pH 2 using 6N hydrochloric acid solution, and then the aqueous layer was separated. 500 Kg of ethyl acetate was added to the obtained aqueous layer, the mixture was neutralized with 20% NaOH solution, and then the organic layer was separated. The organic layer was dried by adding anhydrous magnesium sulfate thereto as a drying agent, filtered to remove the drying agent, and then concentrated. 260 Kg of acetonitrile was added to the resulting residue, and 34.4 Kg of potassium thioacetate was added thereto. The reaction mixture was refluxed for 7 hours and cooled to room temperature (about 25° C.). 400 Kg of ethyl acetate and 240 Kg of purified water were added to the reaction mixture, which was then acidified to pH 2 with 6N hydrochloric acid solution. The aqueous layer was separated, and 120 Kg of ethyl acetate was added thereto. The mixture was neutralized with 20% NaOH solution, and an organic layer was separated. The organic layer was dried by adding anhydrous magnesium sulfate thereto as a drying agent, and filtered to remove the drying agent. The organic layer was concentrated to obtain 44.4 Kg of the title compound (Yield: 74%).

¹H NMR (300 MHz, CDCl₃) δ 2.30 (s, 3H), 3.11 (m, 2H), 3.60 (s, 2H), 3.71 (m, 2H), 4.14 (m, 1H), 7.03 (m, 2H), 7.22 (m, 2H).

The compounds of Examples 17 to 22 were prepared in the same manner as in Example 16, except for using the compounds prepared in Examples 9 to 14 instead of 1-(4-fluorobenzyl)azetidin-3-ol.

### Example 17

### 1-benzyl-3-acetylthio-azetidine

Yield: 64%: ¹H NMR (300 MHz, CDCl₃) δ 2.31 (s, 3H), 3.10 (m, 2H), 3.61 (s, 2H), is 3.70 (m, 2H), 4.15 (m, 1H), 7.26 (m, 5H).

### Example 18

### 1-(4-methylbenzyl)-3-acetylthio-azetidine

Yield: 58%; ¹H NMR (300 MHz, CDCl₃) δ 2.29 (s, 3H), 2.32 (s, 3H), 3.08 (m, 2H), 3.58 (s, 2H), 3.69 (m, 2H), 4.15 (m, 1H), 7.10 (d, 4H).

### Example 19

### 1-(4-methoxybenzyl)-3-acetylthio-azetidine

Yield: 74%; ¹H NMR (300 MHz, CDCl₃) δ 2.27 (s, 3H), 3.05 (m, 2H), 3.53 (s, 2H), 3.66 (m, 2H), 3.77 (s, 3H), 4.12 (m, 1H), 6.81 (d, 2H), 7.17 (d, 2H).

### Example 20

### 1-(2-chlorobenzyl)-3-acetylthio-azetidine

Yield: 69%; ¹H NMR (300 MHz, CDCl₃) δ 2.30 (s, 3H), 3.18 (m, 2H), 3.57 (s, 2H), 3.68 (m, 2H), 4.18 (m, 1H), 7.20 (m, 2H), 7.36 (m, 2H).

### Example 21

### 1-[(1R)-1-phenylethyl]-3-acetylthio-azetidine

Yield: 60%; ¹H NMR (300 MHz, CDCl₃) δ 1.21 (d, 3H), 2.28 (s, 3H), 2.99 (t, 2H), 3.29 (m, 1H), 3.52 (t, 1H), 3.72 (t, 1H), 4.09 (m, 1H), 7.25 (m, 5H).

### Example 22

### 1-(3,4-dimethoxybenzyl)-3-acetylthio-azetidine

Yield: 57%; ¹H NMR (300 MHz, CDCl₃) δ 2.22 (s, 3H), 3.07 (m, 2H), 3.55 (s, 2H), 3.69 (m, 2H), 3.85 (s, 3H), 3.88 (s, 3H), 4.18 (m, 1H), 6.78 (s, 2H), 6.81 (s, 1H).

### Example 23

### 1-(4-fluorobenzyl)-azetidin-3-thiol hydrochloride

1-(4-Fluorobenzyl)-3-acetylthio-azetidine (109 g, 455 mmol) prepared in Example 16 was dissolved in a mixed solvent of methanol (650 ml) and isopropyl alcohol (165 ml), and the reaction mixture was cooled to 0° C. Sodium hydroxide (22 g, 550 mmol) dissolved in methanol (400 ml) was slowly added to the reaction mixture at the same temperature. The reaction mixture was stirred at a temperature ranging from 0 to 5° C. for 30 minutes to complete the reaction. 200 ml of 20% (w/w) hydrochloric acid solution in methanol was added to the reaction mixture, which was then stirred for 30 minutes. The reaction mixture was filtered to remove suspended materials, and the filtrate was distilled under a reduced pressure to completely remove the solvent. Isopropyl ether (1.2 L) was added to the reaction mixture, and the resultant was stirred at a temperature ranging from 0 to 5° C. for 30 minutes. White crystal obtained by filtering the resulting solution was dried in vacuo to obtain 104 g of the title compound as a white powder form (Yield: 98%).

¹H NMR (300 MHz, D₂O) δ 3.8-4.2 (m, 3H), 4.4-4.6 (m, 4H), 7.2 (m, 2H), 7.35 (m, 2H)

### Example 24

### 1-(4-fluorobenzyl)-azetidin-3-thiol sulfate

Using 200 ml of 20% (w/w) sulfuric acid solution in methanol instead of 20% (w/w) hydrochloric acid solution in methanol, the title compound was prepared in the same manner as in Example 23.

Yield: 83%

¹H NMR (300 MHz, D₂O) δ 3.5˜4.3 (m, 3H), 4.2˜4.5 (m, 4H), 7.15 (m, 2H), 7.3 (m, 2H).

### Example 25

### 1-(4-fluorobenzyl)-azetidin-3-thiol phosphate

Using 200 ml of 20% (w/w) phosphoric acid solution in methanol instead of 20% (w/w) hydrochloric acid solution in methanol, the title compound was prepared in the same manner as in Example 23.

Yield: 77%

¹H NMR (300 MHz, D₂O) δ 3.5˜4.3 (m, 3H), 4.2˜4.5 (m, 4H), 7.15 (m, 2H), 7.3 (m, 2H).

Using the compound prepared in Example 17 instead of 1-(4-fluorobenzyl)-3-acetylthio-azetidine, the compounds of Examples 26 to 28 were prepared in the same manners as in Examples 23 to 25, respectively.

### Example 26

### 1-benzylazetidin-3-thiol hydrochloride

Yield: 93%: ¹H NMR (300 MHz, D₂O) δ 3.21 (m, 2H), 3.62 (s, 2H), 3.69 (m, 2H), 5.4 (m, 1H), 7.26 (m, 5H).

### Example 27

### 1-benzylazetidin-3-thiol sulfate

Yield: 82%: ¹H NMR (300 MHz, D₂O) δ 3.22 (m, 2H), 3.62 (s, 2H), 3.70 (m, 2H), 5.42 (m, 1H), 7.25 (m, 5H).

### Example 28

### 1-benzylazetidin-3-thiol phosphate

Yield: 65%: ¹H NMR (300 MHz, D₂O) δ 3.21 (m, 2H), 3.60 (s, 2H), 3.70 (m, 2H), 5.42 (m, 1H), 7.26 (m, 5H).

Using the compound prepared in Example 18 instead of 1-(4-fluorobenzyl)-3-acetylthio-azetidine, the compounds of Examples 29 to 31 were prepared in the same manners as in Examples 23 to 25, respectively.

### Example 29

### 1-(4-methylbenzyl)azetidin-3-thiol hydrochloride

Yield: 94%; ¹H NMR (300 MHz, D₂O) δ 2.28 (s, 3H), 3.02 (m, 2H), 3.24 (m, 2H), 3.60 (s, 2H), 5.02 (m, 1H), 7.09 (d, 4H).

### Example 30

### 1-(4-methylbenzyl)azetidin-3-thiol sulfate

Yield: 87%; ¹H NMR (300 MHz, D₂O) δ 2.25 (s, 3H), 3.02 (m, 2H), 3.22 (m, 2H), 3.58 (s, 2H), 5.02 (m, 1H), 7.05 (d, 4H).

### Example 31

### 1-(4-methylbenzyl)azetidin-3-thiol phosphate

Yield: 78%; ¹H NMR (300 MHz, D₂O) δ 2.27 (s, 3H), 3.00 (m, 2H), 3.25 (m, 2H), 3.60 (s, 2H), 5.02 (m, 1H), 7.10 (d, 4H).

Using the compound prepared in Example 19 instead of 1-(4-fluorobenzyl)-3-acetylthio-azetidine, the compounds of Examples 32 to 34 were prepared in the same manners as in Examples 23 to 25, respectively.

### Example 32

### 1-(4-methoxybenzyl)azetidin-3-thiol hydrochloride

Yield: 88%; ¹H NMR (300 MHz, CDCl₃) δ 2.27 (s, 3H), 3.05 (m, 2H), 3.53 (s, 2H), 3.66 (m, 2H), 3.77 (s, 3H), 4.12 (m, 1H), 6.81 (d, 2H), 7.17 (d, 2H).

### Example 33

### 1-(4-methoxybenzyl)azetidin-3-thiol sulfate

Yield: 73%; ¹H NMR (300 MHz, CDCl₃) δ 2.23 (s, 3H), 3.05 (m, 2H), 3.50 (s, 2H), 3.62 (m, 2H), 3.77 (s, 3H), 4.17 (m, 1H), 6.82 (d, 2H), 7.15 (d, 2H).

### Example 34

### 1-(4-methoxybenzyl)azetidin-3-thiol phosphate

Yield: 54%; ¹H NMR (300 MHz, CDCl₃) δ 2.25 (s, 3H), 3.02 (m, 2H), 3.51 (s, 2H), 3.62 (m, 2H), 3.78 (s, 3H), 4.15 (m, 1H), 6.83 (d, 2H), 7.18 (d, 2H).

Using the compound prepared in Example 20 instead of 1-(4-fluorobenzyl)-3-acetylthio-azetidine, the compounds of Examples 35 to 37 were prepared in the same manners as in Examples 23 to 25, respectively.

### Example 35

### 1-(2-chlorobenzyl)azetidin-3-thiol hydrochloride

Yield: 93%; ¹H NMR (300 MHz, CDCl₃) δ 3.16 (s. 2H), 3.67 (s, 2H), 3.98 (m, 2H), 4.20 (m, 1H), 7.00 (d, 2H), 7.36 (d, 2H).

### Example 36

### 1-(2-chlorobenzyl)azetidin-3-thiol sulfate

Yield: 90%; ¹H NMR (300 MHz, CDCl₃) δ 3.10 (s. 2H), 3.66 (s, 2H), 3.96 (m, 2H), 4.19 (m, 1H), 6.98 (d, 2H), 7.36 (d, 2H).

### Example 37

### 1-(2-chlorobenzyl)azetidin-3-thiol phosphate

Yield: 81%; ¹H NMR (300 MHz, CDCl₃) δ 3.13 (s. 2H), 3.66 (s, 2H), 3.98 (m, 2H), 4.19 (m, 1H), 6.99 (d, 2H), 7.35 (d, 2H).

Using the compound prepared in Example 21 instead of 1-(4-fluorobenzyl)-3-acetylthio-azetidine, the compounds of Examples 38 to 40 were prepared in the same manners as in Examples 23 to 25, respectively.

### Example 38

### 1-[(1R)-1-phenylethyl]azetidin-3-thiol hydrochloride

Yield: 90%; ¹H NMR (300 MHz, CDCl₃) δ 1.42 (d, 3H), 3.80 (m, 2H), 4.08 (m, 2H), 4.42 (m, 1H), 4.44 (m, 1H), 7.39 (m, 5H).

### Example 39

### 1-[(1R)-1-phenylethyl]azetidin-3-thiol sulfate

Yield: 84%; ¹H NMR (300 MHz, CDCl₃) δ 1.48 (d, 3H), 3.85 (m, 2H), 4.08 (m, 2H), 4.43 (m, 1H), 4.62 (m, 1H), 7.39 (m, 5H).

### Example 40

### 1-[(1R)-1-phenylethyl]azetidin-3-thiol phosphate

Yield: 57%; ¹H NMR (300 MHz, CDCl₃) δ 1.49 (d, 3H), 3.82 (bs, 2H), 4.05 (bs, 2H), 4.42 (m, 1H), 4.62 (bs, 1H), 7.42 (m, 5H).

Using the compound prepared in Example 22 instead of 1-(4-fluorobenzyl)-3-acetylthio-azetidine, the compounds of Examples 41 to 43 were prepared in the same manners as in Examples 23 to 25, respectively.

### Example 41

### 1-(3,4-dimethoxybenzyl)azetidin-3-thiol hydrochloride

Yield: 87%; ¹H NMR (300 MHz, CDCl₃) δ 2.95 (m, 2H), 3.55 (s, 2H), 3.60 (m, 1H), 3.68 (m, 2H), 3.84 (s, 3H), 3.86 (s, 3H), 6.78 (s, 2H), 6.80 (s, 1H).

### Example 42

### 1-(3,4-dimethoxybenzyl)azetidin-3-thiol sulfate

Yield: 85%; ¹H NMR (300 MHz, CDCl₃) δ 2.97 (m, 2H), 3.52 (s, 2H), 3.57 (m, 1H), 3.64 (m, 2H), 3.82 (s, 3H), 3.84 (s, 3H), 6.79 (s, 2H), 6.81 (s, 1H).

### Example 43

### 1-(3,4-dimethoxybenzyl)azetidin-3-thiol phosphate

Yield: 75%; ¹H NMR (300 MHz, CDCl₃) δ 2.95 (m, 2H), 3.55 (s, 2H), 3.63 (m, 1H), 3.71 (m, 2H), 3.84 (s, 3H), 3.86 (s, 3H), 6.76 (s, 2H), 6.70 (s, 1H).

### Example 44

### 4-nitrobenzyl (1R,5S,6S)-2-[1-(4-fluorobenzyl)-azetidin-3-yl-thio]-6-[(1R)-1-hydroxyethyl]-1-methylcarbapen-2-em-3-carboxylate hydrochloride

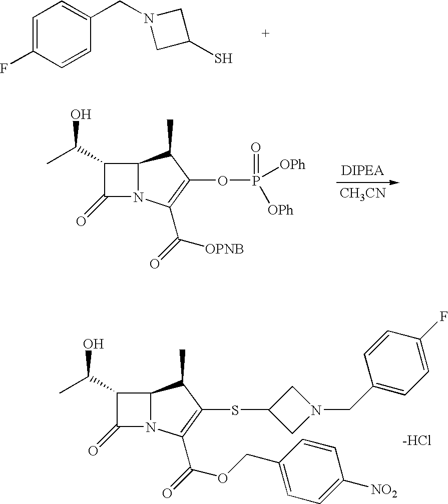

4-Fluorobenzyl-azetidin-3-yl-thiol (25 g, 126 mmol) was dissolved in acetonitrile (400 ml), and the mixture was cooled to −5° C. 4-Nitrobenzyl (1R,5S,6S)-2-(diphenylphosphoryloxy)-6-[(1R)-1-hydroxyethyl]-1-methylcarbapen-2-em-3-carboxylate (50 g, 84 mmol) was added to the mixture at the same temperature.

Diiopropylethylamine (102 ml) was slowly added to the reaction mixture, which was then stirred for 2 hours.

1,600 ml of a mixed solvent of ethyl acetate and purified solvent (1:1, equivalent ratio) was added to the reaction mixture, which was then acidified to pH 3 with a diluted hydrochloric acid solution. The organic layer was separated, and then the aqueous layer was extracted with ethyl acetate. Anhydrous magnesium sulfate was added to the combined organic layer to remove moisture, and then the resultant was concentrated under a reduced pressure. 200 ml of acetone was added to the resulting residue, which was then stirred for 2 hours for crystallization. The reaction mixture was filtered, and the obtained solid was dried to obtain 38 g of the white title compound (Yield: 78%).

¹H NMR (300 MHz, CDCl₃) δ 1.15 (d, J=7.5 Hz, 3H), 1.34 (d, J=6.3 Hz, 3H), 3.11 (m, 2H), 3.23 (m, 2H), 3.72 (m, 2H), 3.95 (m, 1H), 4.21 (m, 2H), 5.34 (q, J=13.8 Hz, 77 Hz, 2H), 6.98 (m, 2H), 7.20 (m, 2H), 7.65 (d, J=8.7 Hz, 2H), 8.22 (d, J=8.7 Hz, 2H).

### Example 46

### 4-nitrobenzyl (1R,5S,6S)-2-[1-(4-fluorobenzyl)-azetidin-3-yl-thio]-6-[(1R)-1-hydroxyethyl]-1-methy Icarbapen-2-em-3-carboxylate sulfate

Using the diluted sulfuric acid solution instead of the diluted hydrochloric acid solution, the title compound was prepared in the same manners as in Example 44.

Yield: 55%;

¹H NMR (300 MHz, CDCl₃) δ 1.15 (d, J=7.5 Hz, 3H), 1.35 (d, J=6.3 Hz, 3H), 3.10 (m, 2H), 3.24 (m, 2H), 3.72 (m, 2H), 3.94 (m, 1H), 4.22 (m, 2H), 5.34 (q, J=13.8 Hz, 77 Hz, 2H), 6.97 (m, 2H), 7.20 (m, 2H), 7.63 (d, J=8.7 Hz, 2H), 8.20 (d, J=8.7 Hz, 2H).

### Example 46

### 4-nitrobenzyl (1R,5S,6S)-2-[1-(4-fluorobenzyl)-azetidin-3-yl-thio]-6-[(1R)-1-hydroxyethyl]-1-methy Icarbapen-2-em-3-carboxylate phosphate

Using the diluted phosphoric acid solution instead of the diluted hydrochloric acid solution, the title compound was prepared in the same manners as in Example 44.

Yield: 51%;

¹H NMR (300 MHz, CDCl₃) δ 1.16 (d, J=7.5 Hz, 3H), 1.34 (d, J=6.3 Hz, 3H), 3.12 (m, 2H), 3.25 (m, 2H), 3.72 (m, 2H), 3.96 (m, 1H), 4.25 (m, 2H), 5.34 (q, J=13.8 Hz, 77 Hz, 2H), 6.98 (m, 2H), 7.19 (m, 2H), 7.64 (d, J=8.7 Hz, 2H), 8.20 (d, J=8.7 Hz, 2H).

### Example 47

### Potassium (1R,5S,6S)-2-[1-(4-fluorobenzyl)-azetidin-3-yl-thio]-6-[(1R)-1-hydroxyethyl]-1-methy Icarbapen-2-em-3-carboxylate

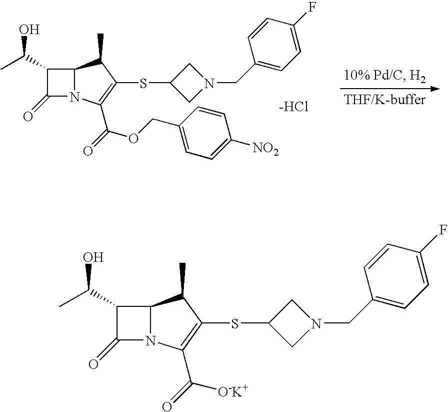

4-Nitrobenzyl (1R,5S,6S)-2-[1-(4-fluorobenzyl)-azetidin-3-yl-thio]-6-[(1R)-1-hydroxyethyl]-1-methylcarbapen-2-em-3-carboxylate hydrochloride (38 g, 66 mmol) prepared in Example 44 was dissolved in a mixed solvent of tetrahydrofuran (380 mL) and a phosphate buffer (pH 7.0, 380 mL), and 10% to palladium/carbon (3.5 g) was added thereto. The resultant was stirred at a temperature ranging from 25 to 30° C. under a hydrogen atmosphere (at atmospheric pressure) for 3 hours, the catalyst was removed by filtration, and the aqueous layer was separated. The separated aqueous layer was washed with ethyl acetate and then lyophilized to obtain 23.1 g of the title compound as a white solid (Yield: 79%, HPLC purity: 98% or higher).

¹H NMR (300 MHz, D₂O) δ 1.17 (d, J=7.3 Hz, 3H), 1.31 (d, J=6.1 Hz, 3H), 3.20 (m, 1H), 3.41 (m, 1H), 3.69 (m, 2H), 4.07 (s, 2H), 4.18 (m, 5H), 7.20 (m, 2H), 7.40 (m, 2H).

1. A process for preparing an acid addition salt of a compound of Formula 3, the process comprising:
(a) reacting a compound of Formula 1 with a compound of Formula 2;
(b) adding a mixed solvent of water and an organic solvent to the reaction mixture prepared in step (a), acidifying the resulting mixture to a pH ranging from 1 to 5, and then separating an organic layer; and
(c) crystallizing by adding an organic solvent to the organic layer obtained in step (b) or its concentrate: 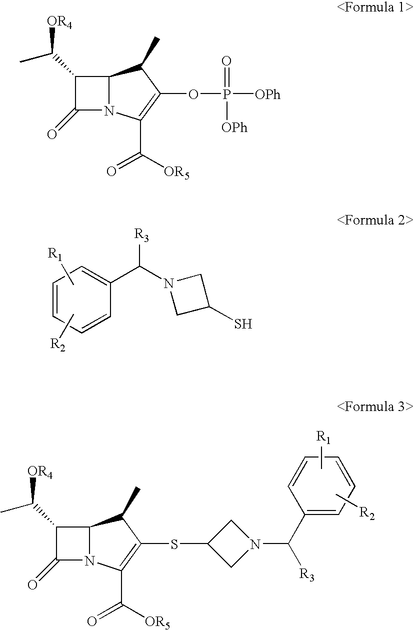
wherein, R₁ and R₂ are each independently hydrogen, C₁-C₃ alkyl, C₁-C₃ alkoxy, halogen, hydroxyl, amino, or trifluoromethyl; R₃ is hydrogen or C₁-C₃ alkyl; R₄ is hydrogen or a hydroxy-protecting group; and R₅ is a carboxy-protecting group.

2. The process of claim 1, wherein the organic solvent used in step (b) is selected from the group consisting of ethyl acetate, tetrahydrofuran, methylene chloride, isopropyl ether, petroleum ether, and diethyl ether.

3. The process of claim 1, wherein the acidification of step (b) is performed using an inorganic acid.

4. The process of claim 3, wherein the inorganic acid is hydrochloric acid, sulfuric acid, or phosphoric acid.

5. The process of claim 1, wherein the organic solvent used in step (c) is ethyl acetate, acetone, toluene, n-hexane, or isopropyl ether.

6. An acid addition salt of the compound of Formula 3: 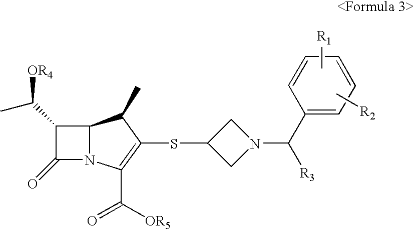
wherein, R₁, R₂, R₃, R₄, and R₅ are the same as defined in claim 1.

7. The acid addition salt of claim 6, wherein the acid addition salt of the compound of Formula 3 is a hydrochloride, a sulfate, or a phosphate of the compound of Formula 3.

8. A process for preparing a compound of Formula 4 or a pharmaceutically acceptable salt thereof, the process comprising deprotecting an acid addition salt of a compound of Formula 3: 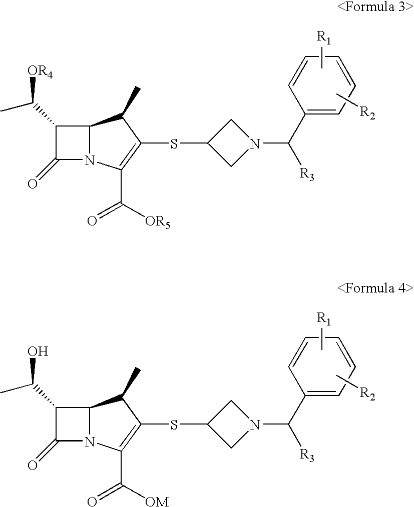
wherein, R₁, R₂, R₃, R₄, and R₅ are the same as defined in claim 1 and M is hydrogen or a counter ion forming a pharmaceutically acceptable salt.

9. The process of claim 8, wherein the acid addition salt of the compound of Formula 3 is prepared according to the process according to claim 1.

10. The process of claim 8, wherein the acid addition salt of the compound of Formula 3 is a hydrochloride, a sulfate, or a phosphate of the compound of Formula 3.

11. A process for preparing an acid addition salt of a compound of Formula 2, the process comprising:
(A) (i) reacting thioacetic acid with a compound of Formula 9, in the presence of triphenylphosphine and diisopropylazodicarboxylate or (ii) subjecting a compound of Formula 9, methanesulfonyl chloride, and alkali metal thioacetate to a reaction;
(B) adding a mixed solvent of water and an organic solvent to the reaction mixture prepared in step (A) or its concentrate, acidifying the resulting mixture to a pH ranging from 1 to 5, and then separating an aqueous layer;
(C) neutralizing the aqueous layer obtained in step (B) and then extracting with an organic solvent to isolate a compound of Formula 10;
(D) reacting the compound of Formula 10 obtained in step (C) with an inorganic base in C₁-C₄ alcohol to deacetylate the compound of Formula 10, and then forming an acid addition salt by adding a solution of an acid in C₁-C₄ alcohol; and
(E) isolating the acid addition salt formed in step (D): 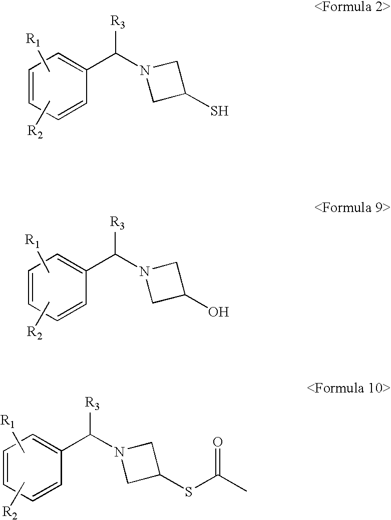
wherein, R₁, R₂ and R₃ are the same as defined in claim 1.

12. The process of claim 11, wherein step (A) is performed by subjecting a compound of Formula 9, methanesulfonyl chloride, and alkali metal thioacetate to a reaction.

13. The process of claim 12, wherein the reaction comprises:
(p) reacting the compound of Formula 9 with methanesulfonyl chloride in the presence of a base;
(q) adding water to the reaction mixture obtained in step (p), acidifying the resultant, separating an aqueous layer, adding an organic solvent to the aqueous layer, neutralizing the resultant, and then separating an organic layer; and
(r) adding an organic solvent to the organic layer obtained in step (q) or its concentrate, and then reacting with alkali metal thioacetate.

14. The process of claim 11, wherein the acidifying in step (B) is performed by acidifying the mixture to a pH ranging from 3 to 4.

15. The process of claim 11, wherein the inorganic base in step (D) is at least one selected from the group consisting of sodium hydroxide (NaOH), potassium hydroxide (KOH), lithium hydroxide (LiOH), and calcium hydroxide (Ca(OH)₂).

16. The process of claim 11, wherein the C₁-C₄ alcohol in step (D) is methanol, ethanol, or isopropanol.

17. The process of claim 11, wherein the acid in step (D) is at least one selected from the group consisting of acetic acid, propionic acid, butyric acid, trifluoroacetic acid, trichloroacetic acid, benzoic acid, nitrobenzoic acid, methanesulfonic acid, diphenyl phosphate, hydrochloric acid, sulfuric acid, phosphoric acid, hydrobromic acid, hydroiodic acid, fluoroboric acid, perchloric acid, and nitrous acid.

18. The process of claim 17, wherein the acid is selected from the group consisting of hydrochloric acid, sulfuric acid, and phosphoric acid.

19. The process of claim 11, wherein the isolation of the acid addition salt in step (E) is performed by crystallizing the acid addition salt from isopropyl ether, ethyl acetate, or n-hexane.

20. The process of claim 11, wherein the compound of Formula 9 is prepared by reacting a compound of Formula 6 with at least one base selected from the group consisting of sodium bicarbonate (NaHCO₃), sodium carbonate (Na₂CO₃), potassium bicarbonate (KHCO₃), potassium carbonate (K₂CO₃), triethylamine (TEA), and diisopropylethylamine (DIPEA): 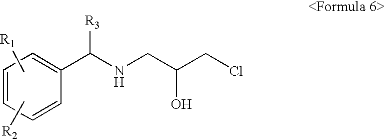
wherein, R₁ and R₂ are each independently hydrogen, C₁-C₃ alkyl, C₁-C₃ alkoxy, halogen, hydroxyl, amino, or trifluoromethyl; and R₃ is hydrogen or C₁-C₃ alkyl.

21. The process of claim 20, wherein the reaction of the compound of Formula 6 and the base is performed in the presence of at least one solvent selected from the group consisting of acetonitrile, toluene, tetrahydrofuran, petroleum ether, and xylene.

22. The process of claim 20, wherein the compound of Formula 6 is prepared by reacting a compound of Formula 5 with epichlorohydrin in water: 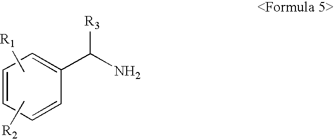
wherein, R₁ and R₂ are each independently hydrogen, C₁-C₃ alkyl, C₁-C₃ alkoxy, halogen, hydroxyl, amino, or trifluoromethyl; and R₃ is hydrogen or C₁-C₃ alkyl.

23. A process for preparing a compound of Formula 6, the process comprising reacting a compound of Formula 5 with epichlorohydrin in water: 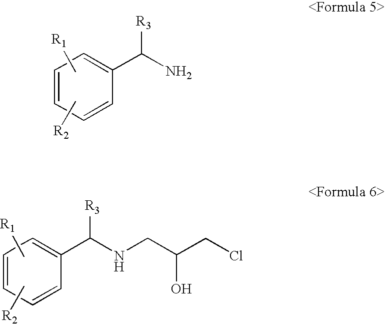
wherein, R₁, R₂ and R₃ are the same as defined in claim 1.

24. An acid addition salt of the compound of Formula 2: 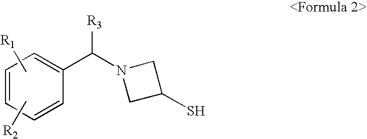
wherein, R₁, R₂, and R₃ are the same as defined in claim 1.

25. The acid addition salt of claim 24, wherein the acid addition salt of the compound of Formula 2 is a hydrochloride, a sulfate, or a phosphate of the compound of Formula 2.
